(19) United States
(12) Patent Application Publication
(10) Pub. No.: US 2001/0006663 A1
(43) Pub. Date: Jul. 5, 2001

(54) ANTIBROMIC MEANS TO INHIBIT/DEODORIZE UNPLEASANT CORPOREAL ODORS

(76) Inventor: LI-CHUAN CHEN, TAOYUAN HSIEN (TW)

Correspondence Address: ROSENBERG KLEIN & BILKER, 3444 ELLICOTT CENTER DRIVE, SUITE 105, ELLICOTT CITY, MD, 21043

(21) Appl. No.: 09/372,618

(22) Filed: Aug. 12, 1999

(30) Foreign Application Priority Data
Aug. 14, 1998 (TW) 87113384

(51) Int. Cl. A61K 9/00

(52) U.S. Cl. 424/400

(57) ABSTRACT

An antibromic means to inhibit/deodorize unpleasant corporeal odors (particularly osmidrosis), in form of a wet paper napkin, a moistened tissue paper or a like flat flexible fibrous substrate with a moisture. In order to improve the cleaning effect and the affinity to a vast variety of pharmaceutical components, the moisture comprises at least two, and preferable three solvents of different polarity and an emulsifier to emulsify the moisture to an emulsion.

This invention relates to antibromic means to inhibit/deodorize the unpleasant corporeal odors (particularly osmidrosis) in form of a flat, flexible fibrous substrate with a moisture. Here the term “fibrous substrate with a moisture” means in particular a disposable wet paper napkin, a moistened tissue paper or the like.

The unpleasant odors of human body is very annoying in many occasions. Particulary in summer when people are thinly and shortly dressed, the unpleasant odors (particularly osmidrotical odor) can more easily dissipate through the thin clothes to the outside atmosphere. Responsible for the unpleasant odors are the ingredients in the sweat or the secretion of axillary glands, which are soon decomposed by bacteria to form stink metabolites such as aldehydes, acids, ammonia and thiols. Therefore the conventional antibromic means are exclusively based on the inhibition of the bacteria (bacteriostasis) to block the decomposition of the ingredients in the sweat and the secretions, using bacteriostats, which are generally applied by means of

1) spraying

2) roll spreading

3) cotton swab

to apply the bacteriostats in form of powder, gel or liquid to the portions where sweat glands are densely distributed. The applied ingredients, in addition to bacteriostats, may also include strong aromatics to outodor the bromidrosis, and astringents to astringe the sweat pores, thus reducing the further sweating and evaporation. All the means of the application can only prevent or retard the worsening of the problem of unpleasant body odor. But none of them can effectively relieve or eliminate the already existing unpleasant odor. In other words, the sweat, dirts and secretion are not removed and still remain at the sweat-stained portions and a part (for example 40%) of the contents has been decomposed by bacteria and are giving off stink odors. The applied agents can only inhibit the further propagation of the bacteria to decompose the remaining (60%) contents and therefore prevent the worsening of the bromidrotic problem, but do not remove the already existing odors. The added strong aromatics may not necessarily outodor the unpleasant odor. Rather the unpleasant bromidrotic odor, when mixed with the aromatics, may be synergistically strengthened and become even more unbearable. If an applied portion (for example the forehead or the axillary portion) sweats too much, the applied bacteriostats have no enough dwelling time for their onset, and may be washed away quickly with the dropping down of the sweat, thus even failing to mitigate the worsening of the bromidrotic problem. Accordingly, the user has to use a towel, a handkerchief, a napkin or a tissue paper to clean the sweat in advance, before he can apply the bacteriostats. Also the above means for application (aerosol atomizer, roller, and cotton swab with container for the bacteriostats) are relatively large in size and rigid (inflexible), as opposed to the flexible handkerchief or wet napkin which can be easily folded up into a small size to put in a handbag or in the pocket.

To solve the bromidrotic problem, one has to prepare two tools: a cleaning means (e.g. a napkin) and an applying means (e.g. an atomizer sprayer), and to perform two procedures: a cleaning procedure to clean the sweat-stained portion, and an applying procedure to apply the bacterostatic agents.

Accordingly, it is an object of this invention to provide an antibromic means to obviate or mitigate the aforesaid problems.

According to the present invention the two tools are combined into a single flexible tool, and the two procedures are carried out in a single step, thus greatly increasing the portability and conveniency.

According to the present invention, the object is achieved by incorporating the antibromic ingredients in a wet napkin or a moistened tissue paper. Thus when one uses a napkin according to this invention to clean the sweat-stained portions, the bacteriostatic substances simultaneously exudes from the napkin and are applied to the treated portions. An user of an antibromic sprayer may have the worry that the sprayer will betray her confidential secret of osmidromis. With the present invention there is no worry, since the package can be made similar to the ordinary wet napkins.

Since the wet napkin can carry bacteriostatic and astringent agents as pharmaceutical ingredients, it can also be used as a provisory first aid means to clean and sterilize the wounds when no band-aid or tincture is available. While the bacteriostatic agent has sterilizing effect, the astringent agent has stanching and styptic effect. Accordingly this invention makes an ideal substitute for first aid. Even if all medical materials are available, this napkin according to this invention can also be used to clean the wound before the formal treatment. Here it functions like a sterilized gauze or cotton. This advantageous application can be printed on the package to instruct the user to exploit its vast potential of utilization.

Another object of this invention is to provide an all around moisture as a good liquid carrier for the pharmaceutical ingredients of the wet paper napkin.

The conventional wet paper napkin generally has a moisture of aquatic medium, which may contain a saponaceous component or a surfactant as cleaning agent. Such an aquatic solvent does not prove fully successful, especially when used as the carrier for the pharmaceutical ingredients in the wet paper napkin according to this invention. Firstly, there are two kinds of glands in the skin, namely sweat glands and sebaceous glands. While the sweat and its dirts can be easily removed by a napkin with an aquatic moisture, the oily secretion of sebaceous glands are not so easily cleaned due to the incompatibility of oil and water. As a result, the cleaning effect is poor. Secondly, since most of the known bacteriostatic agents are not water-soluble, only a minority of the bacteriostats which are hydrophilic can be contained in the aqueous carrier, thus greatly limiting the possibility of the use of pharmacetical ingredients in the wet paper napkin. Furthermore it is desired that the possible largest amount of bacteriostat may exudate when the wet paper napkin contacts with the skin and the pharmaceutical ingredients have the possible longest dwelling time on the skin. However when using a wet paper napkin with a surfactant, the allowable amount of bacteriostats is very small and not easy to stay on the skin, and can be easily carried away by the surfactant. Also the aquatic medium is a poor carrier for astringent or stanching agents (for example, zinc oxide, aluminium alkylchloride, aluminium chloride) which often present in granular or powder form and can only inhomogeneously mixed with water to give a dispersion, which tends to clot or block in the capillaries of the wet paper napkin.

According to this invention, this problem is solved by a moisture containing a three-component-solvent-system with at least two (and preferably three) solvents of different polarity, namely high polarity, moderate polarity (in the case of three solvents), and low polarity (or non-polarity), as carriers for different kinds of pharmaceutical ingredients. Thus the wet paper napkin according to this invention has both hydrophilic and lipophilic property. Any pharmaceutical ingredient can find at least a compatible solvent of its like polarity which offers the required affinity. In order to homogenize the three kinds of solvents, an emulsifying agent must be added in the moisture to give an emulsion.

With the present invention, not only hydrophilic, but hydrophobic (lipophilic) bacteriostats can be effectively incorporated in the wet paper napkin. If necessary, any kinds of adjuvant or additives, for example hidroschetic or anhidrotic components can also be added.

The advantages of the present invention consists in its stronger cleaning effect and its vast affinity to almost all the usable phamaceutical components. As stated before, for the daily job against the annoying corporeal odors, the effective antibromic means of inhibition/deodorization must comprise both the cleaning of the suffered portion and the application of pharmaceutical ingredients. This invention makes it possible to accomplish the cleaning and the application at the same time. Moreover, because of the vast affinity of the three-solvent-system, all the sweat, greasy dirts, or secretion on the skin, either hydrophilic or hydrophobic, can be easily removed. Thus the cleaning effect is much stronger. Also, because of the vast affinity of the three-solvent system, all the usable pharmaceutical ingredients, if desired, can be well absorbed in the wet paper napkin.

Generally the conventional astringent or stanching agents, which have relatively coarse particle sizes and could not be applied in fine form by atomizer, could only be applied by spreading. With the present invention, the difficult astringent or stanching agent can be well emulsified and absorbed in the napkin.

Apart from the strong cleaning effect, the emulsion has a very good affinity to the skin. Thus when a wet paper napkin is used to wipe the skin, a thin film of the emulsion is left on the wiped portion. The emulsion film is not so easily washed away by the further sweating as it is the case of aqueous carrier, thus offering a much longer dwelling time for the pharmaceutical ingredient on the skin. This means a much longer inhibition of the emission of unpleasant corporeal odors or osmidrosis.

As for the solvents for the three-solvent-system of this invention, all the available solvents can be considered, with the following constraints:

1) no toxicity or irritability

2) do not contaminate or stain the clothes,

3) no erosivity to the package and clothes,

4) no unpleasant odors,

Examples of the practical solvents are:

I) High Polar Solvents

malt oil

PPG stearyl ether

Stearyl ether

Lanolin

II) Moderate Polar Solvents

triglyceryl octate/decanoate

isopropyl myristate

isopropyl palmate

III) Low or Non-Polar Solvents

mineral oils

hydrocarbone oils

silicone oils

[☆ Note: the above oils also have decontaminating and emollient effects]

Example of the usable emulsifiers are:

The proportion of each solvent is practically no less than 10% by weight of the totalmoisture.

Examples of practical emulsifiers are:

glycerol monostearate,

polyoxyethylene (2) cetyl ether

polyoxyethylene (20) sorbitan monostearate

sorbitan esters

glyceryl stearate

polyoxyethylene fatty glyceride

which may present in their respective effective concentration.

1. An antibromic means to inhibit/deodorize unpleasant corporeal odors using pharmaceutical ingredients for the inhibition/deodorization, comprising a flat, flexible fibrous substrate with a moisture absorbed therein, said moisture comprising a solvent system carrying said pharmaceutical ingredients, and at least an emulsifier to emulsify said moisture to an emulsion, said solvent system comprising at least two solvents of apparently different polarity, said pharmaceutical ingredients comprise at least a bacteriostat.

2. An antibromic means according to claim 1, wherein said solvent system comprises three solvents, respectively having a high, a moderate and a low polarity.

3. An antibromic means according to claim 1, further comprising an astringent agent in said emulsion.
